(19) United States
(12) Patent Application Publication
(10) Pub. No.: US 2020/0368171 A1
(43) Pub. Date: Nov. 26, 2020

(54) PARTICLES

(71) Applicant: MicroVention, Inc., Aliso Viejo, CA (US)

(72) Inventors: Makoto Harumoto, Aliso Viejo, CA (US); Gregory M. Cruise, Rancho Santa Margarita, CA (US); Michael Kebede, Aliso Viejo, CA (US)

(21) Appl. No.: 16/882,239

(22) Filed: May 22, 2020

Related U.S. Application Data
(60) Provisional application No. 62/852,091, filed on May 23, 2019.

(51) Int. Cl. A61K 9/50; A61K 31/282; A61K 31/337; A61K 33/243; A61K 9/00

(52) U.S. Cl. CPC A61K 9/5026; A61K 31/282; A61K 9/0019; A61K 33/243; A61K 31/337

(57) ABSTRACT

Described are particles configured for intravascular delivery of pharmaceutical agents to a diseased site.

### CROSS REFERENCE TO RELATED APPLICATIONS

This application claims the benefit of Provisional Patent Application Ser. No. 62/852,091 filed May 23, 2019, the entire contents of which are incorporated herein by reference.

### FIELD

Described herein are particles configured for intravascular delivery of pharmaceutical agents to a target site.

### SUMMARY

Described are particles, preparation of particles or methods of delivering particles to intravascular locations or target sites. In some embodiments, these target sites may be diseased and in need of treatment. These particles can deliver pharmaceutical agents to a target site, such as a diseased site. The particles can be delivered to the site, with or without complete cessation of blood flow. Upon delivery to the site, pharmaceutical agents can be released from the particles in a manner as described herein. In some embodiments, that manner can be logarithmic.

Polymer particles described herein can be capable of delivery of pharmaceutical agents as well as embolization. More specifically, the polymer can include at least one monomer, at least one crosslinker, and at least one pharmaceutical agent chemically entrapped within the particle. The pharmaceutical agent can be controllably released from the particle by diffusion and, optionally, by release as the particle degrades.

In one embodiment, the pharmaceutical agent can have a structure

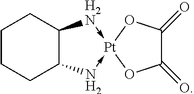

In another embodiment, the pharmaceutical agent can have a structure

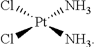

In another embodiment, the pharmaceutical agent can have a structure

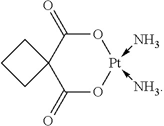

In another embodiment, the pharmaceutical agent can have a structure of

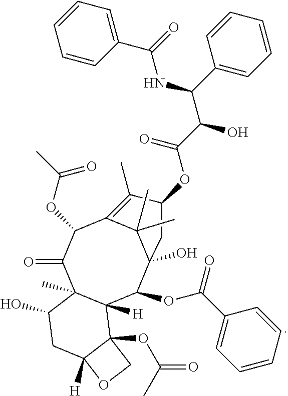 [SMILES: CC(=O)O[C@H]1C(=O)[C@@]2(C)[C@H]([C@H](OC(=O)c3ccccc3)[C@]3(O)C[C@H](OC(=O)[C@H](O)[C@@H](NC(=O)c4ccccc4)c4ccccc4)C(C)=C1C3(C)C)[C@]1(OC(C)=O)CO[C@@H]1C[C@@H]2O]

In another embodiment, the pharmaceutical agent can have a structure

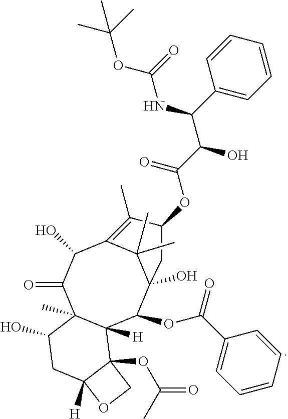 [SMILES: CC(=O)O[C@@]12CO[C@@H]1C[C@H](O)[C@@]1(C)C(=O)[C@H](O)C3=C(C)[C@@H](OC(=O)[C@H](O)[C@@H](NC(=O)OC(C)(C)C)c4ccccc4)C[C@@](O)([C@@H](OC(=O)c4ccccc4)[C@H]21)C3(C)C]

In another embodiment, the pharmaceutical agent can have a structure

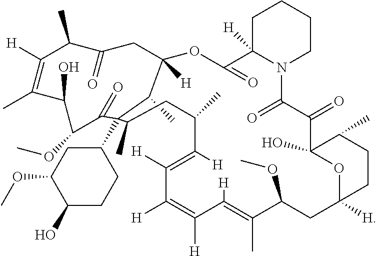

In another embodiment, the pharmaceutical agent can have a structure

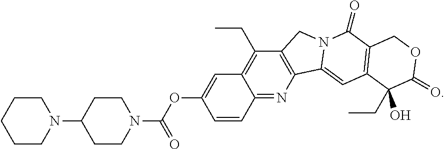 [SMILES: CCc1c2c(nc3ccc(OC(=O)N4CCC(N5CCCCC5)CC4)cc13)-c1cc3c(c(=O)n1C2)COC(=O)[C@]3(O)CC]

In another embodiment, the pharmaceutical agent can have a structure

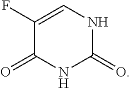

Methods of treating a vessel are also described. These methods can comprise delivering polymer particles as described herein to the vessel to treat the vessel. The polymer particles can be a reaction product of a prepolymer solution including at least one monomer, at least one crosslinker, and at least one pharmaceutical agent. In some embodiments, the at least one pharmaceutical agent is chemically entrapped in the polymer particles.

In some embodiments, the polymer particles can provide a logarithmic elution profile once delivered to the vessel. The logarithmic elution profile can have a sharp initial elution followed by a plateau.

In some embodiments, the polymer particles can elute the at least one pharmaceutical agent for at least 24 hours.

In other embodiments, the polymer particles can elute between about 40 mg and about 60 mg of the at least one pharmaceutical agent per 1 mL of particles.

Methods of making the polymer particles described herein are also described. These particles can elute at least one pharmaceutical agent once delivered These methods of making can include reacting a prepolymer solution including at least one monomer, at least one crosslinker, at least one initiator and at least one pharmaceutical agent in a solvent thereby forming the polymer particles and encapsulating the at least one pharmaceutical agent.

In some embodiments, the methods of making further comprise stirring the prepolymer solution rapidly to create smaller diameter polymer particles or stirring the prepolymer solution slowly to create larger diameter polymer particles.

### BRIEF DESCRIPTION OF THE DRAWINGS

FIG. 1 is a graph illustrating kinetics of oxaliplatin elution from preloaded beads.

FIG. 2 is a graph illustrating kinetics of paclitaxel elution from preloaded beads.

FIG. 3 illustrates oxaliplatin concentration in plasma samples.

FIG. 4 illustrates how livers were harvested. The Figure is labeled as: 1—Upper Right Lateral Liver Lobe (URL), 2—Middle Right Lateral Liver Lobe (MRL), 3—Lower Right Lateral Liver Lobe (LRL), 4—Upper Right Median Liver Lobe (URM), 5—Middle Right Median Liver Lobe (MRM), 6—Lower Right Median Liver Lobe (LRM), 7—Upper Left Median Liver Lobe (ULM), 8—Middle Left Median Liver Lobe (MLM), 9—Lower Left Median Liver Lobe (LLM), 10—Upper Left Lateral Liver Lobe (ULL), 11—Middle Left Lateral Liver Lobe (MLL), and 12—Lower Left Lateral Liver Lobe (LLL).

FIG. 5 illustrates oxaliplatin concentration in tissue samples.

FIG. 6 illustrates paclitaxel concentration in plasma samples.

FIG. 7 illustrates paclitaxel concentration in tissue samples.

FIG. 8 illustrates kinetics of cisplatin elution from the preloaded beads.

FIG. 9 illustrates kinetics of carboplatin elution from the preloaded beads.

FIG. 10 illustrates kinetics of taxotere elution from the preloaded beads.

### DETAILED DESCRIPTION

Polymer particles, methods of preparing these particles and methods of using these particles are described. In some embodiments, the terms particles and beads can be used interchangeably.

The polymer particles described herein can include (i) at least one monomer amenable to polymerization, (ii) at least one crosslinker, and (iii) at least one pharmaceutical agent. In some embodiments, the polymer particles described herein can be formed from a prepolymer solution including (i) at least one monomer amenable to polymerization, (ii) at least one crosslinker, and (iii) at least one pharmaceutical agent.

The monomer(s) and crosslinker(s) can provide physical properties of the particles. Desired physical properties include, but are not limited to, elasticity and robustness to permit delivery through a microcatheter or catheter. The monomer(s) and crosslinker(s) can also control the release of pharmaceutical agents from the particle at least in part by the physical properties exhibited by the particles.

Monomers are low molecular weight chemicals containing a single polymerizable group. The main functions of the monomers, if present, can be to aid in polymerization and to impart specific mechanical properties to the resulting polymer. The monomer(s) can be any molecule with at least a single functionality to incorporate into the resulting polymer and in some embodiments a structure conducive to the desired mechanical property. Monomers can include, but are not limited to, acrylamide, methacrylamide, dimethyl acrylamide, glyercol monomethacrylate, hydroxypropyl acrylate, hydroxyethyl methacrylate, hydroxyethyl acrylate, hydroxybutyl acrylate, methyl methacrylate, tert-butyl acrylate, n-butyl acrylate, n-butyl methacrylate, n-hexyl acrylate, methoxyethyl acrylate, iso-decyl acrylate, 2-ethylhexyl acrylate, stearyl acrylate, N-(tert-octyl) acrylamide, N-octyl methacrylate, combinations thereof, and derivatives thereof. Monomer concentrations can range from about 5% to 50% w/w of a prepolymer solution used to form the polymer.

Crosslinkers, low molecular weight molecules with a plurality of polymerizable moieties, can also be optionally utilized to impart further cross-linking of the resulting particle. The crosslinker can be any molecule with at least two functionalities to incorporate into the resulting polymer and in some embodiments a structure conducive to a desired mechanical property. A crosslinker can be biostable or biodegradable. Biostable crosslinkers can be N,N′-methylenebisacrylamide and ethylene glycol dimethacrylate.

In some embodiments, particles can be designed to dissolve in vivo, or in other words are biodegrade. Linkages unstable in the physiological environment can be introduced in the crosslinker to impart biodegradation by hydrolytic, oxidative, or reductive mechanisms. Linkages susceptible to breakage in a physiological environment include those susceptible to hydrolysis, including esters, thioesters, and carbonates, and those susceptible to enzymatic action, including peptides that are cleaved by matrix metalloproteinases, collagenases, elastases, and cathepsins. Multiple crosslinkers can be utilized to control the rate of degradation in a manner that is not possible with only one. Crosslinker concentrations can be less than 25% of the moles of a prepolymer solution.

In one embodiment, a biodegradable crosslinker has the structure

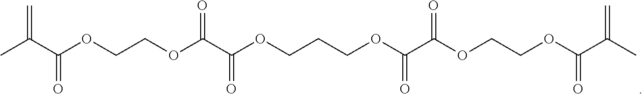 [SMILES: C=C(C)C(=O)OCCOC(=O)C(=O)OCCCOC(=O)C(=O)OCCOC(=O)C(=C)C]

In one embodiment, the biodegradable crosslinker is bis(2-(methacryloyloxy)ethyl) O,O′-(propane-1,3-diyl) dioxalate.

Visualization of particles containing pharmaceutical agents may be desired using medically relevant imaging techniques such as fluoroscopy, computed tomography, or magnetic resonant imaging to permit intravascular delivery and follow-up. Visualization under fluoroscopy can be imparted by the incorporation of solid particles of radiopaque materials such as barium, bismuth, tantalum, platinum, gold, and other dense metals into the polymer or by the incorporation of iodine-containing molecules polymerized into the polymer structure. Visualization agents for fluoroscopy can be barium sulfate and iodine-containing molecules. Visualization under computed tomography imaging can be imparted by incorporation of solid particles of barium or bismuth or by the incorporation of iodine-containing molecules polymerized into the polymer structure. Metals visible under fluoroscopy generally result in beam hardening artifacts that preclude the usefulness of computed tomography imaging for medical purposes. Visualization agents for computed tomography can be barium sulfate and iodine-containing molecules. Concentrations of barium sulfate to render particles visible using fluoroscopic and computed tomography imaging can range from about 30% to about 60% w/w of a prepolymer solution. Concentrations of iodine to render particles visible using fluoroscopic and computed tomography imaging can range from 80 mg l/g of a prepolymer solution.

Visualization under magnetic resonance imaging can be imparted by the incorporation of solid particles of superparamagnetic iron oxide or gadolinium molecules polymerized into the polymer structure. In one embodiment, a visualization agent for magnetic resonance is superparamagnetic iron oxide with a particle size of about 10 microns. Concentrations of superparamagnetic iron oxide particles to render the particles visible using magnetic resonance imaging can range from 0.1% to 1% w/w of a prepolymer solution.

In other embodiments, monomers may be included that impart visualization using medically relevant imaging techniques. Monomers can be halogen-containing molecules polymerized into the embolic structure. Concentrations of iodine, for example, to render the embolic particles visible using fluoroscopic and computed tomography imaging can range from about 80 to about 300 mg l/g of particles in the solvent of the prepolymer solution.

Monomers incorporating visualization characteristics can include one or more halogen atoms. For example, monomers can include 1, 2, 3, 4, 5, 6, 7 or more halogen atoms. In some embodiments, the halogen atoms can be Br or I. In one embodiment, the halogen atoms are I.

In one embodiment, a monomer including a visualization agent or the characteristics of a visualization agent can have a structure:

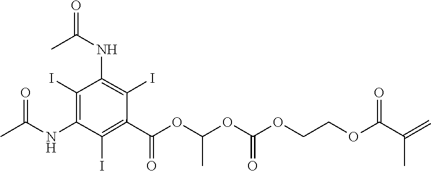 [SMILES: C=C(C)C(=O)OCCOC(=O)OC(C)OC(=O)c1c(I)c(NC(C)=O)c(I)c(NC(C)=O)c1I]

In the above structure, one or more iodine atoms can be replaced by bromine.

In another embodiment, a monomer including a visualization agent or the characteristics of a visualization agent can have a structure:

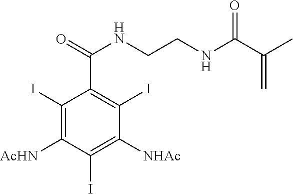 [SMILES: C=C(C)C(=O)NCCNC(=O)c1c(I)c(NC(C)=O)c(I)c(NC(C)=O)c1I]

Again, in the above structure, one or more iodine atoms can be replaced by bromine.

In another embodiment, a monomer including a visualization agent or the characteristics of a visualization agent can have a structure:

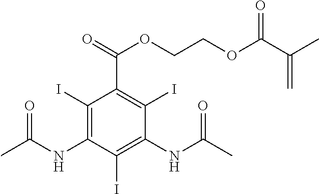 [SMILES: C=C(C)C(=O)OCCOC(=O)c1c(I)c(NC(C)=O)c(I)c(NC(C)=O)c1I]

Again, in the above structure, one or more iodine atoms can be replaced by bromine.

The prepolymer solution can be polymerized by reduction-oxidation, radiation, heat, or any other method known in the art. Radiation cross-linking of the prepolymer solution can be achieved with ultraviolet light or visible light with suitable initiators or ionizing radiation (e.g. electron beam or gamma ray) without initiators. Cross-linking can be achieved by application of heat, either by conventionally heating the solution using a heat source such as a heating well, or by application of infrared light to the monomer solution. The free radical polymerization of the monomer(s) and crosslinker(s) is preferred and requires an initiator to start the reaction. In one embodiment, the cross-linking method utilizes azobisisobutyronitrile (AIBN), 4,4′-Azobis(4-cyanovaleric acid) (ACVA), or another water soluble AIBN derivative (2,2′-azobis(2-methylpropionamidine) dihydrochloride). Other cross-linking agents useful according to the present description can include N,N,N′,N′-tetramethylethylenediamine, ammonium persulfate, benzoyl peroxides, and combinations thereof, including azobisisobutyronitriles. In one embodiment, AIBN can be used as an initiator at a concentration range of about 2% to about 5% w/w.

After the preparation of the particles, they can be optionally dyed to permit visualization during preparation by the physician. Any of the dyes from the family of reactive dyes which bond covalently to particles can be used. Dyes can include, but are not limited to, reactive blue 21, reactive orange 78, reactive yellow 15, reactive blue No. 19 reactive blue No. 4, C.I. reactive red 11, C.I. reactive yellow 86, C.I. reactive blue 163, C.I. reactive red 180, C.I. reactive black 5, C.I. reactive orange 78, C.I. reactive yellow 15, C.I. reactive blue No. 19, C.I. reactive blue 21, any of the color additives approved for use by the FDA part 73, subpart D, or any dye that will irreversibly bond to the polymer matrix of the particle embolic. Alternatively, these dyes can be pre-reacted with free-radical polymerizable monomers containing amines, such as N-(3-aminopropyl methacrylamide) or 2-aminoethyl methacrylate, and then added to the prepolymer solution before polymerization.

The prepolymer solution can be prepared by dissolving the monomer(s), crosslinker(s), pharmaceutical agents, and initiator(s) in a solvent.

Solvents can include, but are not limited to, dimethyl sulfoxide, dimethylformamide, alcohol, acetonitrile, and water. The particles can be prepared by emulsion polymerization.

A non-solvent for the monomer solution, typically mineral oil when the monomer solvent is hydrophilic, and a surfactant can be added to the reaction vessel.

An overhead stirrer can be placed in the reaction vessel. The reaction vessel can then be sealed, and sparged with argon to remove any entrapped oxygen. The initiator component can be added to the reaction vessel and stirring commenced. Additional initiator can be added to the polymerization solution and both can then be added to the reaction vessel, where the stirring suspends droplets of the polymerization solution in the mineral oil. The rate of stirring can affect the size of the particles, with faster stirring producing smaller particles. In other embodiment, stirring more slowly can create larger particles. Stirring rates can range from about 200 to about 1,200 rpm to produce particles with diameters ranging from about 10 to about 1,500 microns. The polymerization can proceed overnight at room temperature.

In some embodiments, stirring the prepolymer solution rapidly to create smaller diameter polymer particles. In other embodiments, stirring the prepolymer solution slowly to create larger diameter polymer particles.

After the polymerization is complete, the polymer particles can be washed to remove any solute, mineral oil, solvent, unreacted monomer(s), untrapped pharmaceutical agents, and unbound oligomers. Washing solutions including any solvent may be utilized, but care should be taken if aqueous solutions are used to wash particles with linkages susceptible to hydrolysis. Additional care should be taken to utilize solvents that do not dissolve the pharmaceutical agent. Washing solutions can include hexanes, dimethylformamide, acetone, alcohols, toluene, xylene, acetonitrile, water with surfactant, water, and saline.

After washing the polymer particles, the polymer particles can be dried to remove any solvent(s). The drying process can proceed overnight by vacuum chamber at room temperature. The lyophilization process can also be utilized for the drying process. After drying, the polymer particles can be packaged into vials or syringes, and sterilized.

Optionally, the washed particles can then be dyed to permit visualization before injection into a microcatheter. A dye bath can be made by dissolving sodium carbonate and the desired dye in water. Particles can be added to the dye bath and stirred. After the dying process, any unbound dye can be removed through copious washing. After dying and additional washing, the microspheres can be packaged into vials or syringes, and sterilized.

The final polymer particle preparation can be delivered to the site to be embolized via a delivery device. A delivery device can be a catheter, a microcatheter, a syringe, or other device that can deliver the particles to a target site.

A radiopaque contrast agent can be thoroughly mixed with the particle preparation in a syringe and injected through a catheter until blood flow is determined to be occluded from the site by interventional imaging techniques.

The pharmaceutical agent(s) can be controllably released from the particle by diffusion and, optionally, by release as the particles degrade. In some embodiments, any pharmaceutical agent can be used that can be entrapped by the polymers described herein.

In one embodiment, the pharmaceutical agent can have a structure

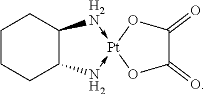

In another embodiment, the pharmaceutical agent can have a structure

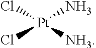

In another embodiment, the pharmaceutical agent can have a structure

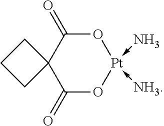

In another embodiment, the pharmaceutical agent can have a structure of

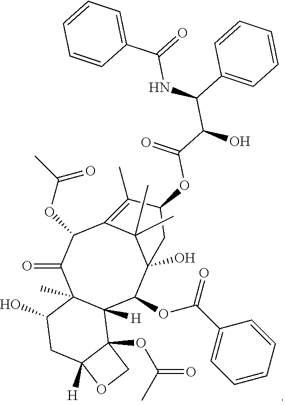 [SMILES: CC(=O)O[C@H]1C(=O)[C@@]2(C)[C@H]([C@H](OC(=O)c3ccccc3)[C@]3(O)C[C@H](OC(=O)[C@H](O)[C@@H](NC(=O)c4ccccc4)c4ccccc4)C(C)=C1C3(C)C)[C@]1(OC(C)=O)CO[C@@H]1C[C@@H]2O]

In another embodiment, the pharmaceutical agent can have a structure

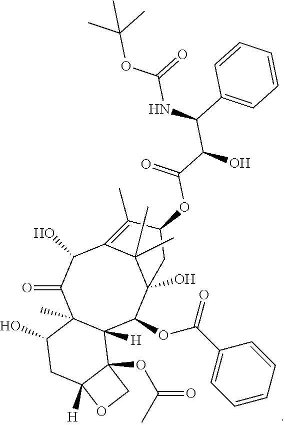 [SMILES: CC(=O)O[C@@]12CO[C@@H]1C[C@H](O)[C@@]1(C)C(=O)[C@H](O)C3=C(C)[C@@H](OC(=O)[C@H](O)[C@@H](NC(=O)OC(C)(C)C)c4ccccc4)C[C@@](O)([C@@H](OC(=O)c4ccccc4)[C@H]21)C3(C)C]

In another embodiment, the pharmaceutical agent can have a structure

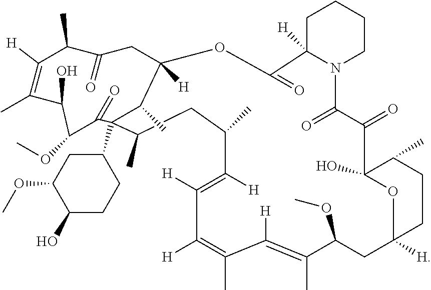

In another embodiment, the pharmaceutical agent can have a structure

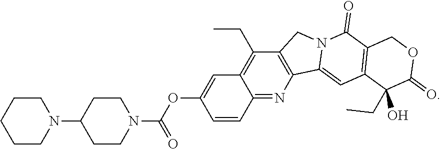 [SMILES: CCc1c2c(nc3ccc(OC(=O)N4CCC(N5CCCCC5)CC4)cc13)-c1cc3c(c(=O)n1C2)COC(=O)[C@]3(O)CC]

In another embodiment, the pharmaceutical agent can have a structure

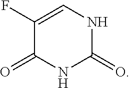

In some embodiments, the pharmaceutical agent can be oxaliplatin, paclitaxel, cisplatin, carboplatin, taxotere, a derivative thereof, a pharmaceutically acceptable salt thereof, and/or a combination thereof.

The particles describe herein can elute the pharmaceutical agent once delivered or implanted. In some embodiments, this elution can be logarithmic with a sharp initial elution followed by a plateau through the remainder of the elution.

In some embodiments, the prepolymer solutions described herein can include acrylamide, N,N-dimethyl acrylamide, a crosslinker, and oxaliplatin. Particles formed from such a prepolymer solution can exhibit a logarithmic elution curve over about a 24 hour period for oxaliplatin. This curve can include a sharp concentration increase in the first two hours followed by a plateau through the 24 hour period.

In another embodiment, the prepolymer solutions described herein can include acrylamide, N,N-dimethylacrylamide, a crosslinker, and paclitaxel. Particles formed from such a prepolymer solution can exhibit a logarithmic elution curve over about a 3 day period for paclitaxel. This curve can include a sharp concentration increase in the first seven hours followed by a plateau through the 3 day period.

In some embodiments, the prepolymer solutions described herein can include acrylamide, N,N-dimethyl acrylamide, a crosslinker, and cisplatin. Particles formed from such a prepolymer solution can exhibit a logarithmic elution curve over about a 24 hour period for oxaliplatin. This curve can include a sharp concentration increase in the first two hours followed by a plateau through the 24 hour period.

In some embodiments, the prepolymer solutions described herein can include acrylamide, N,N-dimethyl acrylamide, a crosslinker, and carboplatin. Particles formed from such a prepolymer solution can exhibit a logarithmic elution curve over about a 6 day period for carboplatin. This curve can include a sharp concentration increase in the first 120 hours followed by a plateau through the 6 day period.

In some embodiments, the prepolymer solutions described herein can include acrylamide, N,N-dimethyl acrylamide, a crosslinker, and taxotere. Particles formed from such a prepolymer solution can exhibit a logarithmic elution curve over about a 24 hour period for taxotere. This curve can include a sharp concentration increase in the first two hours followed by a plateau through the 24 hour period.

In some embodiments, the particles describe herein can elute between about 40 mg and about 60 mg, about 50 mg and about 60 mg, or about 40 mg and about 50 mg per 1 mL of particles. In some embodiments, the particles describe herein can elute about 55 mg per 1 mL of particles.

### EXAMPLE 1

### Preparation of a Particle Containing a Pharmaceutical Agent

Mineral oil (500 mL) is added to a sealed jacketed-reaction vessel equipped with an overhead stirring element and a heating element maintained at 85° C. The vessel is sparged with argon for 1-2 hours while mixing. A prepolymer solution is prepared by dissolving 1.0 g acrylamide, 1.0 g N,N-dimethyl acrylamide, 0.3 g crosslinker from Example 5, 0.14 g of 4-4′-azobis(4-cyanovaleric acid) and 1.61 g of oxaliplatin, in 4.6 g of dimethylsulfoxide. Azobisisobutyronitrile (0.83 g) is added to the reaction vessel and overhead stirring is increased to 350 rpm. After approximately 10 min, an aliquot of SPAN® 80 (1.5 mL) is added to the mineral oil and allowed to mix. The prepolymer solution is added to the reaction vessel and the resulting suspension was allowed to polymerize for an hour before the heat was turned off. The resulting solution is mixed in the reaction vessel overnight.

After polymerization is complete, the overhead stirring is decreased to 275 rpm and 500 mL of hexane is added into the reaction vessel to begin the washing process. After stirring for 10 minutes, the beads are allowed to settle for 5 minutes before the hexane/mineral oil is removed via a chemical transfer pump. This process is repeated for a total of four hexane washes.

After washing, the beads are separated by size using a sieving process. The contents of the reaction vessel are poured over a stack of sieves ordered largest to smallest, from top to bottom, along with an aliquot of hexane. Once all the particles had been sorted, they are collected and placed in bottles according to their size.

After sieving, the particles are dehydrated to extend their shelf life. Under stirring, the beads are placed in 100% acetone. The acetone is exchanged five times after at least 20 minutes of mixing between each exchange. Subsequently, the acetone is poured out and any solvent remaining is removed via a vacuum oven.

### EXAMPLE 2

### Preparation of a Particle Containing a Pharmaceutical Agent

Mineral oil (500 mL) is added to a sealed jacketed-reaction vessel equipped with an overhead stirring element and a heating element maintained at 85° C. The vessel is sparged with argon for 1-2 hours while mixing. A prepolymer solution is prepared by dissolving 0.5 g acrylamide, 0.5 g N,N-dimethylacrylamide, 0.3 g crosslinker from Example 5, 0.14 g of 4,4′-azobis(4-cyanovaleric acid) and 1.0 g of paclitaxel in 4.6 g of dimethylsulfoxide. Azobisisobutyronitrile (0.83 g) is added to the reaction vessel and overhead stirring increased to 350 rpm. After approximately 2 min, an aliquot of SPAN® 80 (1.5 mL) is added to the mineral oil and allowed to mix. The prepolymer solution is added to the reaction vessel and the resulting suspension is allowed to polymerize for an hour before the heat is turned off. The resulting solution is mixed in the reaction vessel overnight.

After polymerization is complete, the overhead stirring is decreased to 275 rpm and 500 mL of hexane is added into the reaction vessel to begin the washing process. After stirring for 10 minutes, the beads are allowed to settle for 5 minutes before the hexane/mineral oil is removed via a chemical transfer pump. This process is repeated for a total of four hexane washes.

After washing, the beads are separated by size using a sieving process. The contents of the reaction vessel are poured over a stack of sieves ordered largest to smallest, from top to bottom, along with an aliquot of hexane. Once all the particles had been sorted, they are collected and placed in bottles according to their size. Excess hexane is poured out and any solvent remaining is removed via a vacuum oven.

### EXAMPLE 3

### In Vitro Elution of Pharmaceutical Agents from Particles

Into a 50 mL centrifuge tube, 50 mg of dry oxaliplatin preloaded particles are added, as prepared in Example 1. The beads are suspended in 50 mL of phosphate buffered saline (PBS) and placed in a 37° C. oven. At 30 minutes, 1, 2, and 4 hours, 10 mL of the supernatant is pipetted out and placed in a 15 mL centrifuge tube for ICP-MS analysis. The remaining PBS is poured out and the beads are re-suspended with fresh PBS and placed back at 37° C. The last sample is taken after 24 hours. Additionally, dry oxaliplatin preloaded particles are evaluated to calculate loading.

Samples are prepared for ICP-MS analysis to measure the platinum concentration in the beads by mixing a sample portion (100 μL) with 1 mL nitric acid and 3 mL hydrochloric acid, then digested using a microwave. After cooling, internal standards are added and digestates are diluted to a final mass of 100 g using high-purity water.

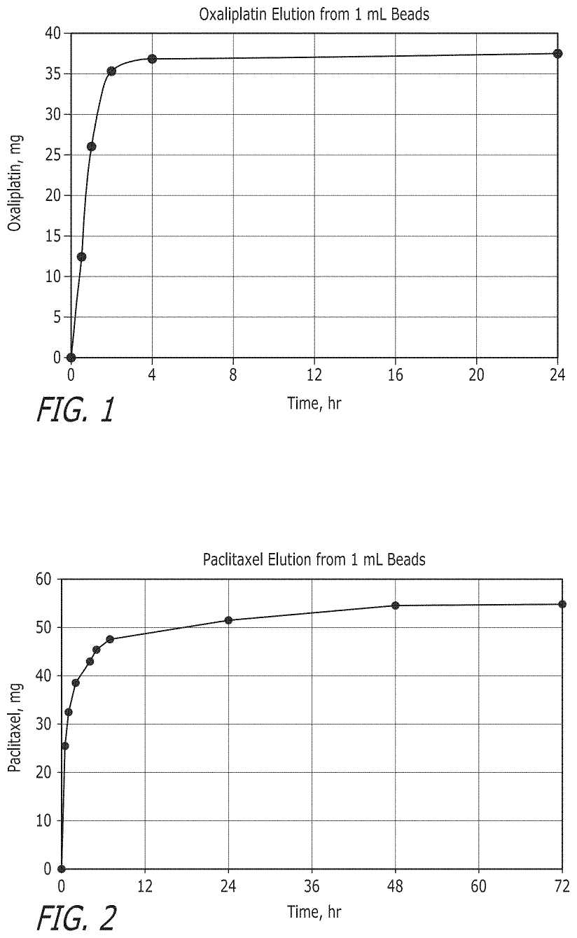

The kinetics of oxaliplatin elution from the preloaded beads are shown in FIG. 1. The elution curve is close to a logarithmic curve over the period of 24 hours with a sharp increase of 35mg of oxaliplatin being eluted in the first 2 hours before plateauing through the 24-hour period.

### EXAMPLE 4

### In Vitro Elution of Pharmaceutical Agents from Particles

Into a 50 mL centrifuge tube, 50 mg of dry paclitaxel preloaded particles are added, as prepared in Example 2. The beads are suspended in 50 mL of a dissolution medium made up of a 45:55 acetonitrile/10 mM potassium phosphate buffer solution with an adjusted pH of 4.5 and placed in a 37° C. oven. At 30 minutes, 1, 2, 4, 5, and 7 hours, 10 mL of the supernatant is pipetted out and placed in a 15 mL centrifuge tube. The remaining dissolution medium is poured out and the beads are re-suspended with a fresh 50 mL of the dissolution medium and placed back at 37° C. Additional samples are taken at 24 hours, 48 hours, and 72 hours, at which point the beads are fully dissolved in the dissolution medium.

The concentration of paclitaxel in each sample is determined using an Agilent 1100 HPLC system. The chromatographic analysis is performed with an Agilent Extended-C18 column (4.6 mm×50 mm, 3.5 μm). The mobile phases are delivered at 1 mL/min consisted of 65% acetonitrile and 35% 5% acetonitrile in water. The injection volume is 5 μL and the wavelength of the ultraviolet detector is 227 nm. The calibration curve is prepared from 0.5 to 200 ppm of paclitaxel. The amount of paclitaxel released and relative percentage are calculated from the concentration data.

The kinetics of paclitaxel elution from the preloaded beads are shown in FIG. 2. The elution curve obtained is close to a logarithmic curve over the period of 3 days with a sharp increase of 48 mg within the first 7 hours before eventually plateauing through the 3-day period. The total amount of paclitaxel eluted during the first 3 days is 55 mg per 1 mL of beads.

### EXAMPLE 5

### Biodegradable Crosslinker

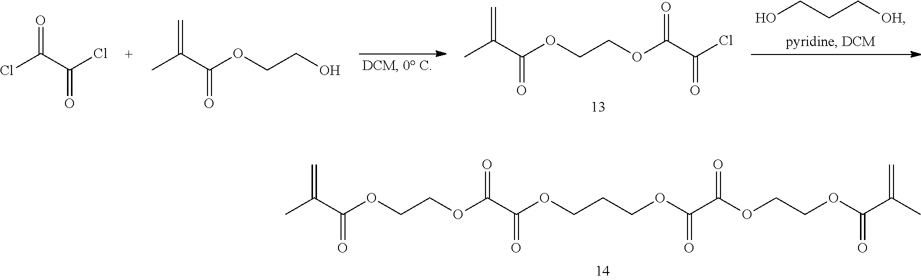

Synthesis of 2-(Methacryloxy)Ethyl Oxalyl Monochloride, 13: An oven-dried 100 mL three-neck round bottom flask is purged under argon. The flask is fitted with a stir bar and an addition funnel. To the flask is added oxalyl chloride (20 g, 158 mmol) and anhydrous DCM (15 mL) sequentially. To the addition funnel is added 2-hydroxyethyl methacrylate (HEMA) (16 g, 123 mmol). The flask is cooled in an ice bath and added HEMA dropwise to the reaction. After the addition is finished, the flask is left stirring in the ice bath for 1 hour. The flask is pulled out of the ice bath and kept stirring for 1 hour. To work up, the DCM and oxalyl chloride are removed on a rotary evaporator. Moisture is avoided from here on. The product is a greenish liquid. It does not move on a silica TLC plate and has a strong UV absorption.

Synthesis of 14: An oven-dried 50 mL three-neck round bottom flask is purged under argon. 2-(Methacryloxy)ethyl oxalyl monochloride (13, 12 g, 54.4 mmol) and anhydrous DCM (25.4 mL) are added to the reaction flask. Pyridine (5.08 g, 64.2 mmol) and 1,3-propanediol (1.88 g, 24.7 mmol) are added sequentially to the flask. To work up, we began with filtering off the white precipitate. Then, filtrate is washed with 5% citric acid (50 mL×2). The DCM fraction is washed with saturated sodium chloride (50 mL) and dried over Na₂SO₄. The solvent is removed under reduced pressure to give the crude product as a thick yellowish liquid. The product is obtained after a flash column separation (normal phase, ethyl acetate/hexanes) as a clear liquid.

### EXAMPLE 6

### Preparation of a Particle Containing a Pharmaceutical Agent

Mineral oil (500 mL) is added to a sealed jacketed-reaction vessel equipped with an overhead stirring element and a heating element maintained at 85° C. The vessel is sparged with argon for 1-2 hours while mixing. A prepolymer solution is prepared by dissolving 0.325 g acrylamide, 0.325 g N,N-dimethylacrylamide, 0.65 g crosslinker from Example 5, 0.14 g of 4,4′-azobis(4-cyanovaleric acid) and 1.0 g of paclitaxel in 4.6 g of dimethylsulfoxide. Azobisisobutyronitrile (0.83 g) is added to the reaction vessel and overhead stirring increased to 350 rpm. After approximately 2 min, an aliquot of SPAN® 80 (1.5 mL) is added to the mineral oil and allowed to mix. The prepolymer solution is added to the reaction vessel and the resulting suspension is allowed to polymerize for an hour before the heat is turned off. The resulting solution is mixed in the reaction vessel overnight.

After polymerization is complete, the overhead stirring is decreased to 275 rpm and 500 mL of hexane is added into the reaction vessel to begin the washing process. After stirring for 10 minutes, the beads are allowed to settle for 5 minutes before the hexane/mineral oil is removed via a chemical transfer pump. This process is repeated for a total of 4 hexane washes, followed by xylene wash and at toluene wash.

After washing, the beads are separated by size using a sieving process. The contents of the reaction vessel are poured over a stack of sieves ordered largest to smallest, from top to bottom, along with an aliquot of toluene. Once all the particles had been sorted, they are collected and placed in bottles according to their size.

The beads were then washed with water. Excess solvents are poured out and any solvent remaining is removed via a vacuum oven.

### EXAMPLE 7

### Pharmacokinetics of Pharmaceutical Accents from Particles

About 20 mL of phosphate buffered saline was added to about 125 mg of dried and sterilized oxaliplatin loaded beads, prepared in Example 1, to be expanded to about 1 mL beads. After pouring out the excess phosphate buffered saline, the beads were mixed with about 4 mL of 50:50 saline:contrast solution. Then the beads were delivered to the left liver in porcine pig through microcatheter.

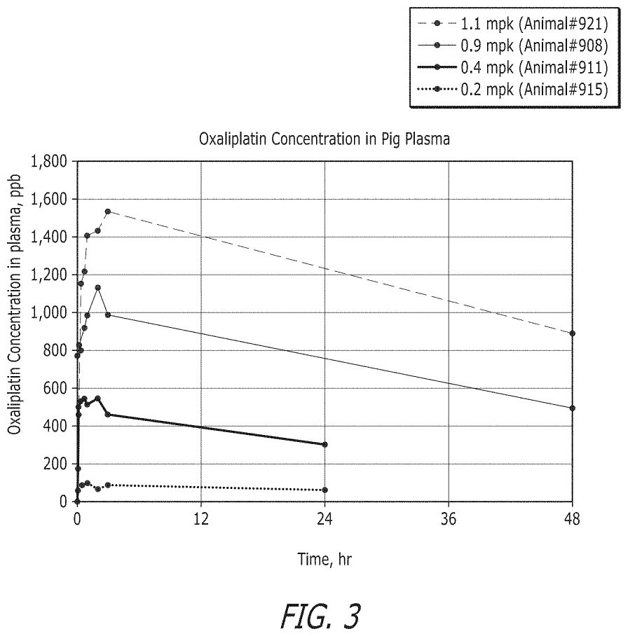

The blood samples were collected before beads injection and at 5, 10, 20, 40, 60, 120, 180 min and post embolization, and processed to plasma. Then the plasma samples were digested and, the oxaliplatin concentration was measured by ICP-MS. The oxaliplatin concentration in plasma samples were represented in FIG. 3.

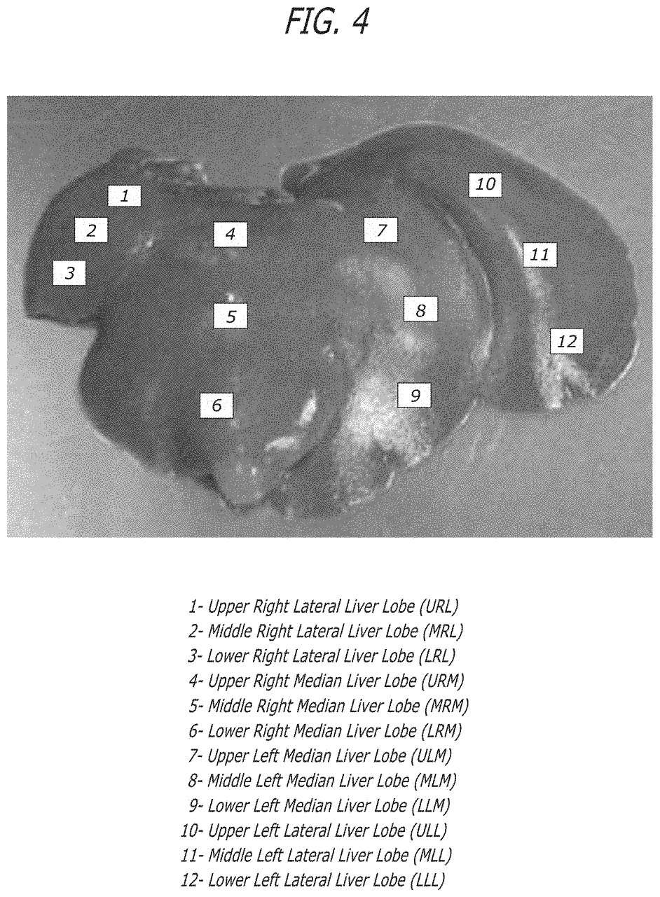

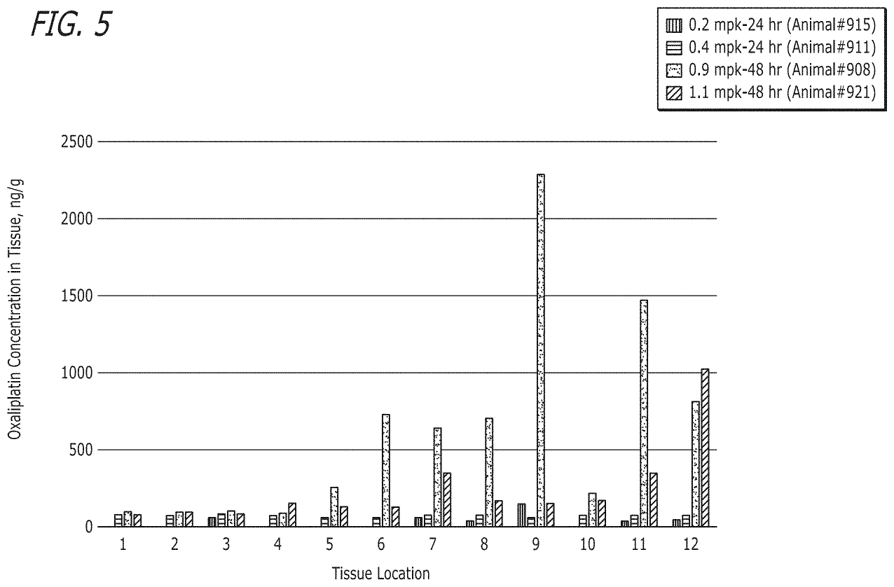

The livers were harvested per the picture in FIG. 4, and the tissue samples from each liver lob were collected. Then the tissue samples were digested, and the oxaliplatin concentration was measured by ICP-MS. The oxaliplatin concentration in tissue samples were represented in FIG. 5.

### EXAMPLE 8

### Pharmacokinetics of Pharmaceutical Accents from Particles

About 20 mL of phosphate buffered saline was added to about 250 mg to 330 mg of dried and sterilized paclitaxel loaded beads, prepared in Example 6, to be expanded to about 1 mL beads. After pouring out the excess phosphate buffered saline, the beads were mixed with about 4 mL of 50:50 saline:contrast solution. Then the beads were delivered to the left liver in porcine pig through microcatheter.

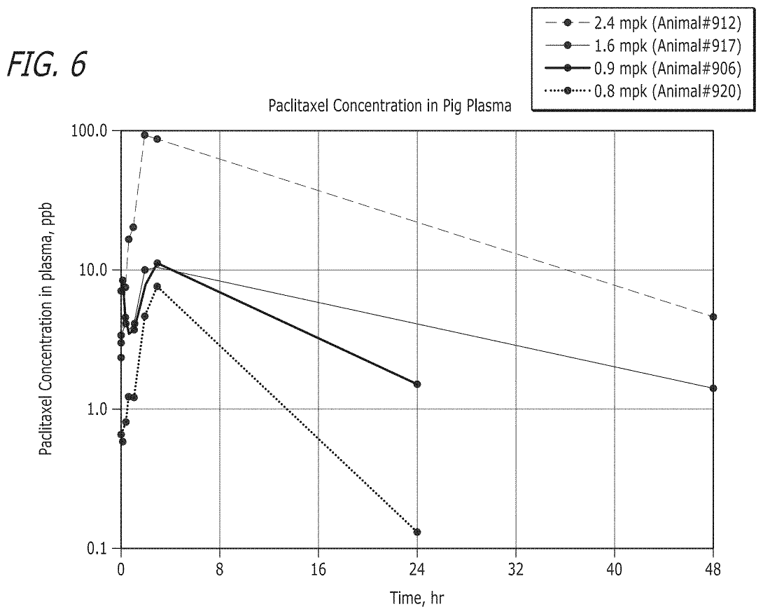

The blood samples were collected before beads injection and at 5, 10, 20, 40, 60, 120, 180 min and post embolization, and processed to plasma. Then the paclitaxel concentration in the plasma samples were measured by LC-MS/MS. The paclitaxel concentration in plasma samples were represented in FIG. 6.

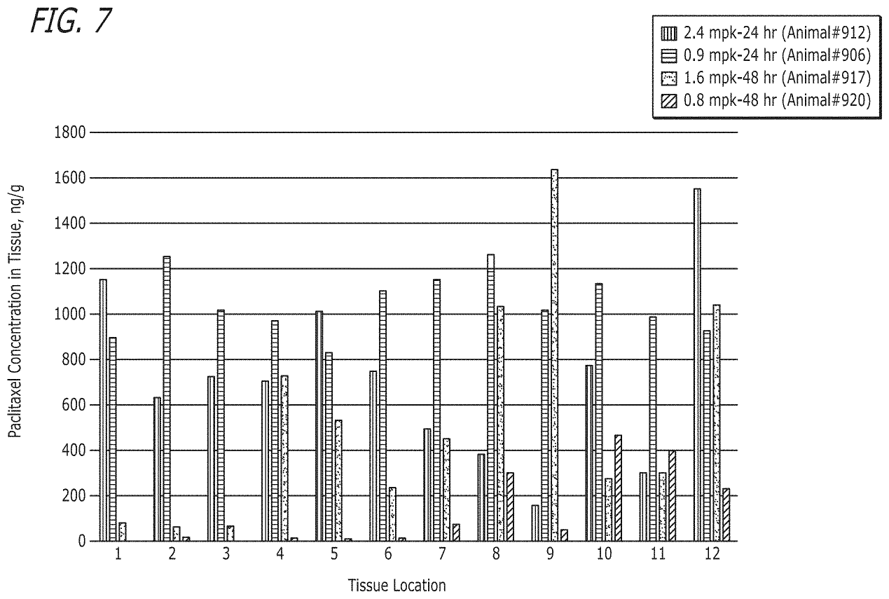

The livers were harvested per the picture in FIG. 4, and the tissue samples from each liver lob were collected. After homogenizing the tissue samples, the paclitaxel concentration in the tissue samples were measured by LC-MS/MS. The paclitaxel concentration in tissue samples were represented in FIG. 7.

### EXAMPLE 9

### Preparation of a Particle Containing a Pharmaceutical Agent

Mineral oil (500 mL) is added to a sealed jacketed-reaction vessel equipped with an overhead stirring element and a heating element maintained at 85° C. The vessel is sparged with argon for 1-2 hours while mixing. A prepolymer solution is prepared by dissolving 0.325 g acrylamide, 0.325 g N,N-dimethyl acrylamide, 0.65 g crosslinker from Example 5, 0.14 g of 4-4′-azobis(4-cyanovaleric acid) and 1.0 g of cisplatin, in 4.6 g of dimethylsulfoxide. Azobisisobutyronitrile (0.83 g) is added to the reaction vessel and overhead stirring is increased to 350 rpm. After approximately 10 min, an aliquot of SPAN® 80 (1.5 mL) is added to the mineral oil and allowed to mix. The prepolymer solution is added to the reaction vessel and the resulting suspension was allowed to polymerize for an hour before the heat was turned off. The resulting solution is mixed in the reaction vessel overnight.

After polymerization is complete, the overhead stirring is decreased to 275 rpm and 500 mL of hexane is added into the reaction vessel to begin the washing process. After stirring for 10 minutes, the beads are allowed to settle for 5 minutes before the hexane/mineral oil is removed via a chemical transfer pump. This process is repeated for a total of four hexane washes.

After washing, the beads are separated by size using a sieving process. The contents of the reaction vessel are poured over a stack of sieves ordered largest to smallest, from top to bottom, along with an aliquot of hexane. Once all the particles had been sorted, they are collected and placed in bottles according to their size.

After sieving, the particles are transferred to centrifuge tubes before being left in the vacuum oven over night.

### EXAMPLE 10

### In Vitro Elution of Pharmaceutical Agents from Particles

Into a 50 mL centrifuge tube, 50 mg of dry cisplatin preloaded particles are added, as prepared in Example 9. The beads are suspended in 50 mL of phosphate buffered saline (PBS) and placed in a 37° C. oven. At 30 minutes, 1, 2, 4.5 and 6 hours, 10 mL of the supernatant is pipetted out and placed in a 15 mL centrifuge tube for ICP-MS analysis. The remaining PBS is poured out and the beads are re-suspended with fresh PBS and placed back at 37° C. The last sample is taken after 24 hours.

Samples are prepared for ICP-MS analysis to measure the platinum concentration in the beads by mixing a sample portion (2 mL) with 10 mL of 2% nitric acid/0.5% hydrochloric acid solution, and Internal standard.

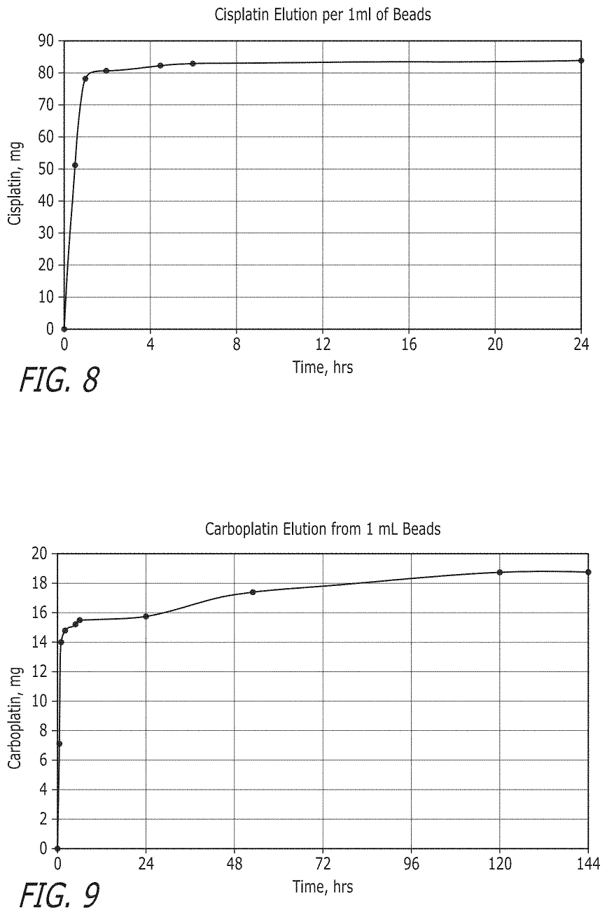

The kinetics of cisplatin elution from the preloaded beads are shown in FIG. 8. The elution curve is close to a logarithmic curve over the period of 24 hours with a sharp increase of 80 mg of cisplatin being eluted in the first 2 hours before plateauing through the 24-hour period.

### EXAMPLE 11

### Preparation of a Particle Containing a Pharmaceutical Agent

Mineral oil (500 mL) is added to a sealed jacketed-reaction vessel equipped with an overhead stirring element and a heating element maintained at 85° C. The vessel is sparged with argon for 1-2 hours while mixing. A prepolymer solution is prepared by dissolving 0.325 g acrylamide, 0.325 g N,N-dimethyl acrylamide, 0.65 g crosslinker from Example 5, 0.14 g of 4-4′-azobis(4-cyanovaleric acid) and 0.345 g of carboplatin, in 4.6 g of dimethylsulfoxide. Azobisisobutyronitrile (0.83 g) is added to the reaction vessel and overhead stirring is increased to 350 rpm. After approximately 10 min, an aliquot of SPAN® 80 (1.5 mL) is added to the mineral oil and allowed to mix. The prepolymer solution is added to the reaction vessel and the resulting suspension was allowed to polymerize for an hour before the heat was turned off. The resulting solution is mixed in the reaction vessel overnight.

After polymerization is complete, the overhead stirring is decreased to 275 rpm and 500 mL of hexane is added into the reaction vessel to begin the washing process. After stirring for 10 minutes, the beads are allowed to settle for 5 minutes before the hexane/mineral oil is removed via a chemical transfer pump. This process is repeated for a total of four hexane washes.

After washing, the beads are separated by size using a sieving process. The contents of the reaction vessel are poured over a stack of sieves ordered largest to smallest, from top to bottom, along with an aliquot of hexane. Once all the particles had been sorted, they are collected and placed in bottles according to their size.

After sieving, the particles are dehydrated to extend their shelf life. Under stirring, the beads are placed in 100% acetone. The acetone is exchanged 5 times after at least 20 minutes of mixing between each exchange. Subsequently, the acetone is poured out and any solvent remaining is removed via a vacuum oven.

### EXAMPLE 12

### In Vitro Elution of Pharmaceutical Agents from Particles

Into a 50 mL centrifuge tube, 50 mg of dry carboplatin preloaded particles are added, as prepared in Example 11. The beads are suspended in 50 mL of phosphate buffered saline (PBS) and placed in a 37° C. oven. At 30 minutes, 1, 2, 5, 6, 24, 53 and 120 hours, 10 mL of the supernatant is pipetted out and placed in a 15 mL centrifuge tube for ICP-MS analysis. The remaining PBS is poured out and the beads are re-suspended with fresh PBS and placed back at 37° C. The last sample is taken after 144 hours.

Samples are prepared for ICP-MS analysis to measure the platinum concentration in the beads by mixing a sample portion (2 mL) with 10 mL of 2% nitric acid/0.5% hydrochloric acid solution, and an internal standard.

The kinetics of carboplatin elution from the preloaded beads are shown in FIG. 9. The elution curve is close to a logarithmic curve over the period of 144 hours with a sharp increase of 18 mg of carboplatin being eluted in the first 120 hours before plateauing through the 144-hour period.

### EXAMPLE 13

### Preparation of a Particle Containing a Pharmaceutical Agent

Mineral oil (500 mL) is added to a sealed jacketed-reaction vessel equipped with an overhead stirring element and a heating element maintained at 85° C. The vessel is sparged with argon for 1-2 hours while mixing. A prepolymer solution is prepared by dissolving 0.325 g acrylamide, 0.325 g N,N-dimethyl acrylamide, 0.65 g crosslinker from Example 5, 0.14 g of 4-4′-azobis(4-cyanovaleric acid) and 1.1 g of taxotere (docetaxel), in 4.6 g of dimethylsulfoxide. Azobisisobutyronitrile (0.83 g) is added to the reaction vessel and overhead stirring is increased to 350 rpm. After approximately 10 min, an aliquot of SPAN® 80 (1.5 mL) is added to the mineral oil and allowed to mix. The prepolymer solution is added to the reaction vessel and the resulting suspension was allowed to polymerize for an hour before the heat was turned off. The resulting solution is mixed in the reaction vessel overnight.

After polymerization is complete, the overhead stirring is decreased to 275 rpm and 500 mL of hexane is added into the reaction vessel to begin the washing process. After stirring for 10 minutes, the beads are allowed to settle for 5 minutes before the hexane/mineral oil is removed via a chemical transfer pump. This process is repeated for a total of four hexane washes.

After washing, the beads are separated by size using a sieving process. The contents of the reaction vessel are poured over a stack of sieves ordered largest to smallest, from top to bottom, along with an aliquot of hexane. Once all the particles had been sorted, they are collected and placed in bottles according to their size.

After sieving, the particles are transferred to centrifuge tubes before being left in the vacuum oven over night.

### EXAMPLE 14

### In Vitro Elution of Pharmaceutical Agents from Particles

Into a 50 mL centrifuge tube, 50 mg of dry taxotere preloaded particles are added, as prepared in Example 13. The beads are suspended in 50 mL of a dissolution medium made up of a 45:55 acetonitrile/deionized water and placed in a 37° C. oven. At 30 minutes, 2, 4, and 5.5 hours, 10 mL of the supernatant is pipetted out and placed in a 15 mL centrifuge tube. The remaining dissolution medium is poured out and the beads are re-suspended with a fresh 50 mL of the dissolution medium and placed back at 37° C. Additional sample is taken at 24 hours, at which point the beads are fully dissolved in the dissolution medium.

The concentration of taxotere in each sample is determined using an Agilent 1100 HPLC system. The chromatographic analysis is performed with an Agilent Extended-C18 column (4.6 mm×50 mm, 3.5 μm). The mobile phases are delivered at 1 mL/min consisted of 65% acetonitrile and 35% 5% acetonitrile in water. The injection volume is 5 μL and the wavelength of the ultraviolet detector is 227 nm. The calibration curve is prepared from 0.5 to 200 ppm of paclitaxel. The amount of taxotere released and relative percentage are calculated from the concentration data.

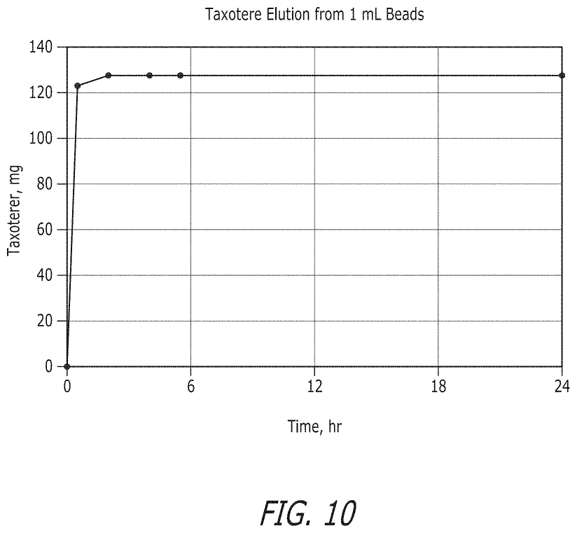

The kinetics of taxotere elution from the preloaded beads are shown in FIG. 10. The elution curve is close to a logarithmic curve over the period of 24 hours with a sharp increase of 127 mg of taxotere being eluted in the first 2 hours before plateauing through the 24-hour period.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

We claim:

1. An embolic particle comprising:
a reaction product of a prepolymer solution including:
at least one monomer selected from acrylamide, methacrylamide, dimethyl acrylamide, glyercol monomethacrylate, hydroxypropyl acrylate, hydroxyethyl methacrylate, hydroxyethyl acrylate, hydroxybutyl acrylate, methyl methacrylate, tert-butyl acrylate, n-butyl acrylate, n-butyl methacrylate, n-hexyl acrylate, methoxyethyl acrylate, iso-decyl acrylate, 2-ethylhexyl acrylate, stearyl acrylate, N-(tert-octyl) acrylamide, N-octyl methacrylate, or a combinations thereof,
at least one crosslinker selected from N,N′-methylenebisacrylamide, ethylene glycol dimethacrylate, 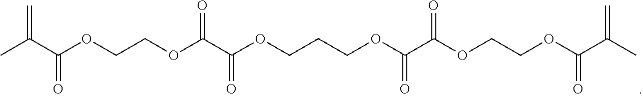 [SMILES: C=C(C)C(=O)OCCOC(=O)C(=O)OCCCOC(=O)C(=O)OCCOC(=O)C(=C)C]
or a combination thereof, and
at least one pharmaceutical agent,
wherein the at least one pharmaceutical agent is chemically entrapped in the embolic particle.

2. The embolic particle of claim 1, wherein the at least one crosslinker is biostable.

3. The embolic particle of claim 1, wherein the at least one crosslinker is biodegradable.

4. The embolic particle of claim 1, wherein the prepolymer solution further includes a solvent.

5. The embolic particle of claim 4, wherein the solvent is dimethyl sulfoxide, dimethylformamide, alcohol, acetonitrile, or water.

6. The embolic particle of claim 1, wherein the prepolymer solution further includes a visualization agent.

7. The embolic particle of claim 1, including two monomers selected from acrylamide, methacrylamide, dimethyl acrylamide, glyercol monomethacrylate, hydroxypropyl acrylate, hydroxyethyl methacrylate, hydroxyethyl acrylate, hydroxybutyl acrylate, methyl methacrylate, tert-butyl acrylate, n-butyl acrylate, n-butyl methacrylate, n-hexyl acrylate, methoxyethyl acrylate, iso-decyl acrylate, 2-ethylhexyl acrylate, stearyl acrylate, N-(tert-octyl) acrylamide, or N-octyl methacrylate.

8. The embolic particle of claim 1, wherein the at least one crosslinker is N,N′-methylenebisacrylamide, ethylene glycol dimethacrylate, or a combination thereof.

9. The embolic particle of claim 1, wherein the at least one crosslinker is

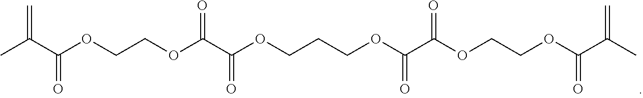 [SMILES: C=C(C)C(=O)OCCOC(=O)C(=O)OCCCOC(=O)C(=O)OCCOC(=O)C(=C)C]

10. The embolic particle of claim 1, wherein the prepolymer solution further includes an initiator.

11. The embolic particle of claim 10, wherein the initiator is azobisisobutyronitrile, 4,4′-azobis(4-cyanovaleric acid), 2,2′-azobis(2-methylpropionamidine) dihydrochloride, N,N,N′,N′-tetramethylethylenediamine, ammonium persulfate, benzoyl peroxides, or a combination thereof.

12. The embolic particle of claim 1, wherein the at least one pharmaceutical agent selected from 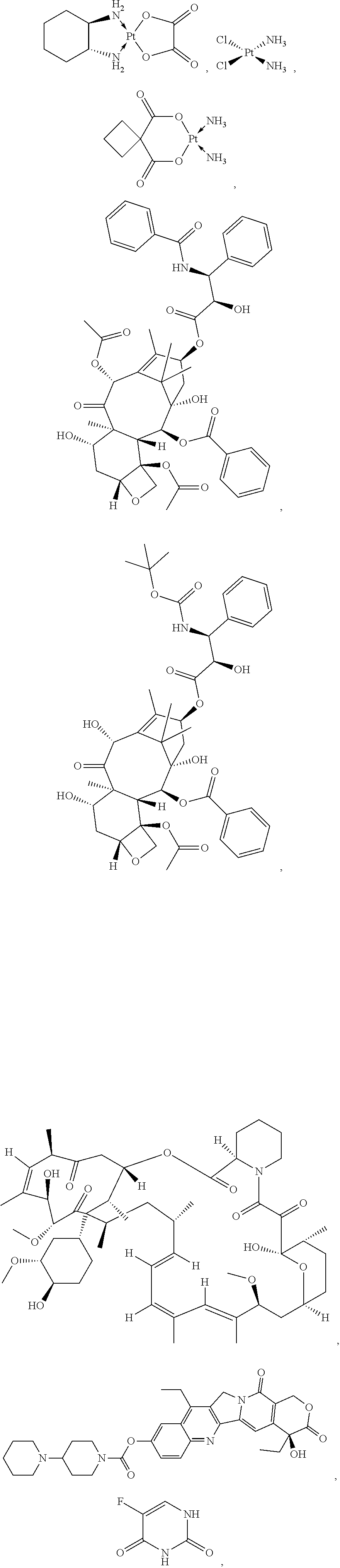
or a combination thereof

13. A method of treating a vessel comprising:
delivering polymer particles to the vessel to treat the vessel, wherein the polymer particles are a reaction product of a prepolymer solution including at least one monomer, at least one crosslinker, and at least one pharmaceutical agent,
wherein the at least one pharmaceutical agent is chemically entrapped in the polymer particles.

14. The method of claim 13, wherein the polymer particles provide a logarithmic elution profile once delivered to the vessel.

15. The method of claim 14, wherein the logarithmic elution profile has a sharp initial elution followed by a plateau.

16. The method of claim 13, wherein the polymer particles elute the at least one pharmaceutical agent for at least 24 hours.

17. The method of claim 13, wherein the polymer particles elute between about 40 mg and about 60 mg of the at least one pharmaceutical agent per 1 mL of particles.

18. A method of making polymer particles that elute at least one pharmaceutical agent, the method comprising:
reacting a prepolymer solution including at least one monomer, at least one crosslinker, at least one initiator and at least one pharmaceutical agent in a solvent, and
forming the polymer particles and encapsulating the at least one pharmaceutical agent.

19. The method of claim 17 further comprising stirring the prepolymer solution rapidly to create smaller diameter polymer particles.

20. The method of claim 17 further comprising stirring the prepolymer solution slowly to create larger diameter polymer particles.
